(19) United States
(12) Patent Application Publication
(10) Pub. No.: US 2014/0336337 A1
(43) Pub. Date: Nov. 13, 2014

(54) Modifying Polymeric Materials By Amines

(71) Applicant: Dow Corning Corporation, MIDLAND, MI (US)

(72) Inventors: Michael Wolfgang Backer, Mainz (DE); Thomas Chaussee, Fontaines Saint Martin (FR); Olivier Debever, Lembeek (BE)

(21) Appl. No.: 14/362,656

(22) PCT Filed: Dec. 7, 2012
(86) PCT No.: PCT/EP2012/074738

§ 371 Date: Jun. 4, 2014

(30) Foreign Application Priority Data
Dec. 8, 2011 (GB) 1121132.3

(51) Int. Cl. C08C 19/22; C07F 3/06; C07D 295/088

(52) U.S. Cl. CPC C08C 19/22; C07D 295/088; C07F 3/06
USPC 525/375; 544/398; 556/134

(57) ABSTRACT

This invention relates to the modification of polymeric materials containing reactive carbon-to-carbon unsaturation and to amines, including piperazines, which are used in such modification. A polymeric material containing carbon-to-carbon bonds can be modified by crosslinking or to make it susceptible to crosslinking.

This invention relates to the modification of polymeric materials containing reactive carbon-to-carbon unsaturation and to amines, including piperazines and aziridines, which are used in such modification. A polymeric material containing carbon-to-carbon bonds can be modified by crosslinking or to make it susceptible to crosslinking.

Many of the amines, including piperazines, which are used in the modification of materials containing carbon-to-carbon unsaturation are new compounds. Thus the invention also relates to substituted piperazines and to their preparation, and to other substituted amines and to their preparation.

An article in Russian Journal of Applied Chemistry; Volume 82, Issue 5, Pages 928-930; Journal 2009; by V. M. Farzaliev, M. T. Abbasova, A. A. Ashurova, G. B. Babaeva, N. P. Ladokhina and Ya. M. Kerimova describes the preparation of bis(alkoxymethyl)piperazines by condensation of piperazine with formaldehyde and aliphatic alcohols.

GB1203036 describes gelatin hardeners of the formula R′OCH2N(R)(CH2)nN(R)CH2OR′ wherein R and R′ are alkyl groups of 1-4 carbon atoms and n is 2 to 10. U.S. Pat. No. 3,379,707 describes a curable polymer composition comprising chlorinated polyethylene and a curing agent which may be selected from a group comprising 2,2′-dithio-bisbenzimidazole and N,N′-diphenyl-p-phenylene diamine.

GB1214451 describes a polymer comprising units containing piperazine derived ring.

HU 180661 describes poly[(piperazine-N′N′-bismethyl)-(1,2-propylen-bisdithiocarbamate)].

A process according to one aspect of the invention for modifying a polymeric material containing carbon-to-carbon unsaturation is characterised in that the polymeric material is treated with a compound (I) containing in its molecule at least two moieties of the formula

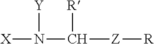

wherein X represents a hydrocarbyl or substituted hydrocarbyl group having 1 to 20 carbon atoms; Y represents hydrogen or a hydrocarbyl or substituted hydrocarbyl group having 1 to 20 carbon atoms; R′ represents hydrogen or a hydrocarbyl or substituted hydrocarbyl group having 1 to 20 carbon atoms; Z represents oxygen or sulphur; and R represents a hydrocarbyl or substituted hydrocarbyl group having 1 to 20 carbon atoms, at least one of the groups X and R being a multivalent substituted hydrocarbyl group linking two or more

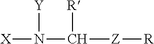

moieties.

The amine compounds (I) of the invention, including the substituted piperazines are capable of crosslinking a polymeric material containing carbon-to-carbon unsaturation. We believe that upon heating, for example to the temperatures used in elastomer processing, the etheramine moiety of (I) forms a very reactive species which reacts with the C═C bonds present in the polymeric material through [2+3] cycloaddition.

Thus in one process according to the invention the polymeric material and the amine compound (I) are heated together at a temperature of 120 to 200° C., whereby the polymeric material is crosslinked by the substituted piperazine.

In an alternative process according to the invention the polymeric material and the amine compound (I) are mixed at a temperature of 0 to 120° C. and subsequently heated at a temperature of 120 to 200° C. to crosslink the polymeric material. When mixing at an elevated temperature below 120° C., there may be some modification of the polymeric material which can be detected via infra-red spectroscopy, for example at least some of the amine compound (I) may be bonded to the polymeric material without substantial crosslinking.

In the compound (I), the groups X and Y can both be substituted hydrocarbyl groups linking the same two

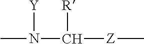

moieties to form a piperazine ring. The compound of formula (I) can thus be a substituted piperazine of the formula [R—Z—(CHR′-Pip-CHR′—Z—R″—Z)ₙ—CH2-Pip-CH2-Z]ₘ—R* where each R represents a hydrocarbyl or substituted hydrocarbyl group having 1 to 20 carbon atoms; each R′ represents hydrogen or a hydrocarbyl or substituted hydrocarbyl group having 1 to 8. carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=1 to 6; and R* is the residue of an alcohol, thiol, polyol or polythiol having at least m hydroxyl or thiol groups.

In one preferred type of substituted piperazine of the formula [R—Z—(CHR′-Pip-CHR′—Z—R″—Z)ₙ—CH2-Pip-CH2-Z]ₘ—R* used for modifying a polymeric material containing carbon-to-carbon unsaturation, n=0, m=1, and each atom Z in the substituted piperazine represents an oxygen atom, that is the substituted piperazine has the formula R—O—CHR′-Pip-CHR′—O—R, in which each R represents a hydrocarbyl or substituted hydrocarbyl group having 1 to 20 carbon atoms.

Such a substituted piperazine has the formula R—O—CHR′-Pip-CHR′—O—R can be prepared by reacting a piperazine with an aldehyde of the formula R′CHO and an alcohol of the formula ROH.

In the substituted piperazine of the formula R—O—CHR′-Pip-CHR′—OR, each group R preferably represents a hydrocarbyl group having 1 to 8 carbon atoms, for example an alkyl group such as an ethyl, methyl, butyl, hexyl or 2-ethylhexyl, an aryl group such as phenyl or an aralkyl group such as benzyl. Most preferably each R represents an ethyl group. The alcohol ROH may be released during crosslinking of a polymer, and ethanol is the most environmentally friendly compound among the alcohols.

The aldehyde which is reacted with the piperazine and the alcohol is preferably formaldehyde to form a substituted piperazine of the formula R—O—CH₂-Pip-CH₂—O—R, although other aldehydes such as acetaldehyde can be used. The piperazine reagent is preferably unsubstituted at the 2-, 3-, 5- and 6-positions, although the piperazine ring can alternatively be substituted in any or all of the 2-, 3-, 5-, or 6-positions by a substituent which does not react with an aldehyde or an alcohol such as an alkyl substituent, for example by one or more methyl groups.

For the substituted piperazines of the formula:

[R—Z—(CHR′-Pip-CHR′—Z—R″—Z)ₙ—CH2-Pip-CH2-Z]ₘ—R*

in which n=0 and m=1, it is preferred that each atom Z in the substituted piperazine represents an oxygen atom rather than a sulphur atom, to avoid release of a volatile thiol on crosslinking.

An alternative preferred type of substituted piperazine has the formula:

[R—Z—(CHR′-Pip-CHR′—Z—R″—Z)ₙ—CH2-Pip-CH2-Z]ₘ—R*,

where each R represents a hydrocarbyl or substituted hydrocarbyl group having 2 to 20 carbon atoms; each R′ represents hydrogen or a hydrocarbyl or substituted hydrocarbyl group having 1 to 8 carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=2 to 6; and R* is the residue of a polyol or polythiol having at least m hydroxyl or thiol groups. Such a substituted piperazine can be prepared by reacting piperazine with an aldehyde of the formula R′CHO and a polyol or polythiol of the formula R*(ZH)ₘ.

These substituted piperazines of the formula:

[R—Z—(CHR′-Pip-CHR′—Z—R″—Z)ₙ—CH2-Pip-CH2-Z]ₘ—R*,

where m=2 to 6 and R* is the residue of a polyol or polythiol having at least m hydroxyl or thiol groups are new compounds. The invention thus includes a substituted piperazine of the formula:

[R—Z—(CHR′-Pip-CHR′—Z—R″—Z)ₙ—CH2-Pip-CH2-Z]ₘ—R*,

where each R represents a hydrocarbyl or substituted hydrocarbyl group having 2 to 20 carbon atoms; each R′ represents hydrogen or a hydrocarbyl or substituted hydrocarbyl group having 1 to 8 carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=2 to 6; and R* is the residue of a polyol or polythiol having at least m hydroxyl or thiol groups.

Each piperazine ring of the novel substituted piperazines of the formula:

[R—Z—(CHR′-Pip-CHR′—Z—R″—Z)ₙ—CH2-Pip-CH2-Z]ₘ—R*,

where m=2 to 6 and R* is the residue of a polyol or polythiol having at least m hydroxyl or thiol groups is preferably unsubstituted at the 2-, 3-, 5- and 6-positions, although the piperazine ring can alternatively be substituted in any or all of the 2-, 3-, 5-, or 6-positions by a substituent which does not react with an aldehyde or an alcohol such as an alkyl substituent. Preferred substituted piperazines of the formula [R—Z—(CHR′-Pip-CHR′—Z—R″—Z)ₙ—CH2-Pip-CH2-Z]ₘ—R*, where m=2 to 6 and R* is the residue of a polyol or polythiol having at least m hydroxyl or thiol groups thus have the formula:

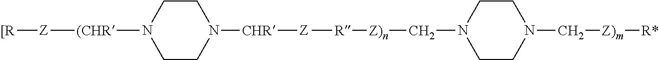

where R, Z, R′, R″, R*, n and m are defined as above.

A process according to the invention for preparing a substituted piperazine of the formula:

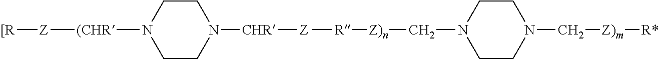

where each R represents a hydrocarbyl or substituted hydrocarbyl group having 1 to 20 carbon atoms; each R′ represents hydrogen or a hydrocarbyl or substituted hydrocarbyl group having 1 to 8 carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=2 to 6; and R* is the residue of an alcohol or polyol having at least m hydroxyl groups, comprises reacting piperazine with an aldehyde of the formula R′CHO and a polyol or polythiol of the formula R*(ZH)_{z}, where z=2 to 6 and z is greater than or equal to m.

Examples of polyols which can be reacted with an aldehyde, for example formaldehyde, and piperazine include diols such as ethylene glycol, di- and tri-ethylene glycol and polyethyleneglycol of varying chain lengths, propyleneglycol, di- and tripropyleneglycol and polypropyleneglycol of varying chain lengths, butane-1,3-diol and butane-1,4-diol, neopentyl glycol, hexane-1,6-diol, isosorbide, 1,4-cyclohexanedimethanol, bisphenol-A, hydroquinone or resorcinol lengthened with ethylene oxide and propylene oxide; triols such as trimethylolpropane, glycerol, trimethylolethane, 2-hydroxymethylbutane-1,4-diol, any of which can be lengthened with ethylene oxide or propylene oxide., and higher polyols such as pentaerythritol and di-pentaerythritol.

The piperazine and the aldehyde can if desired be reacted with a mixture of a polyol of the formula R*(OH)_{z} where R* is the residue of a polyol having z hydroxyl groups, where z=2 to 6, and an alcohol of the formula ROH, where R represents a hydrocarbyl group having 1 to 20 carbon atoms, to form a substituted piperazine of the formula:

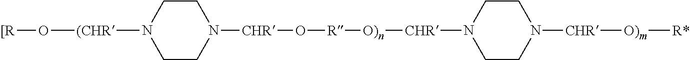

where z is greater than or equal to m.

Alternatively the compound (I) can be a compound containing in its molecule at least two moieties of the formula:

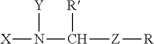

wherein X represents a multivalent substituted hydrocarbyl group linking two or more

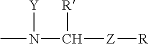

groups; Y represents a hydrocarbyl or substituted hydrocarbyl group having 1 to 20 carbon atoms; Z represents oxygen; and none of Y, R and R′ is a multivalent substituted hydrocarbyl group linking two or more

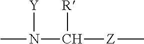

moieties.

The compound (I) may for example have the formula:

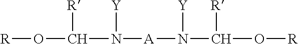

wherein each Y represents a hydrocarbyl or substituted hydrocarbyl group having 1 to 20 carbon atoms; each R′ represents hydrogen or a hydrocarbyl or substituted hydrocarbyl group having 1 to 20 carbon atoms; each R represents a hydrocarbyl or substituted hydrocarbyl group having 1 to 20 carbon atoms, and none of Y, R and R′ is a multivalent substituted hydrocarbyl group linking two or more

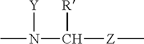

moieties; and A represents a divalent group. A may for example represent a divalent organic group having 2 to 20 carbon atoms, for example an alkylene group.

An alternative preferred divalent group A is a metal carboxylate group of the formula:

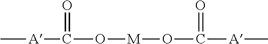

wherein each A′ represents an alkylene group having 1 to 6 carbon atoms; and M represents a divalent metal ion. The compound (I) may thus be of the formula:

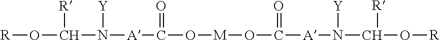

wherein Y represents a hydrocarbyl or substituted hydrocarbyl group having 1 to 20 carbon atoms; each R′ represents hydrogen or a hydrocarbyl or substituted hydrocarbyl group having 1 to 20 carbon atoms; each R represents a hydrocarbyl or substituted hydrocarbyl group having 1 to 20 carbon atoms, and none of Y, R and R′ is a multivalent substituted hydrocarbyl group linking two or more

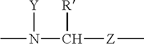

moieties.

A preferred divalent metal M is zinc. Alternative divalent metals include magnesium, copper and iron.

The compound of the formula:

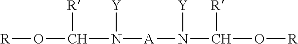

can in general be prepared by reacting a diamine of the formula Y—NH-A-NH—Y, where A represents a divalent group and Y represents a hydrocarbyl or substituted hydrocarbyl group having 1 to 20 carbon atoms, with an aldehyde of the formula R′CHO, where R′ represents hydrogen or a hydrocarbyl or substituted hydrocarbyl group having 1 to 20 carbon atoms, and an alcohol of the formula ROH where R represents a hydrocarbyl or substituted hydrocarbyl group having 1 to 20 carbon atoms, none of Y, R and R′ being a multivalent substituted hydrocarbyl group linking two or more

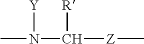

moieties.

The compounds of the formula:

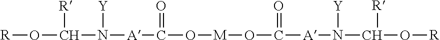

as described above are new compounds. The invention thus includes a metal carboxylate of the formula:

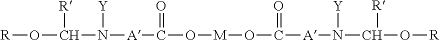

wherein each A′ represents an alkylene group having 1 to 6 carbon atoms; M represents a divalent metal ion; R represents a hydrocarbyl or substituted hydrocarbyl group having 1 to 20 carbon atoms; R′ represents hydrogen or a hydrocarbyl or substituted hydrocarbyl group having 1 to 20 carbon atoms; Y represents a hydrocarbyl or substituted hydrocarbyl group having 1 to 20 carbon atoms; and none of Y, R and R′ is a multivalent substituted hydrocarbyl group linking two or more

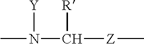

moieties.

The invention also includes a process for the preparation of a metal carboxylate of the formula M(-O—C(═O)-A′-N(Y)—CH(R′)—O—R)ₘ where m is the valence of the metal M wherein each A′ represents an alkylene group having 1 to 6 carbon atoms; M represents a metal ion of charge m; and Y represents a hydrocarbyl or substituted hydrocarbyl group having 1 to 20 carbon atoms; is reacted with an aldehyde of the formula R′CHO wherein R′ represents hydrogen or a hydrocarbyl or substituted hydrocarbyl group having 1 to 20 carbon atoms and an alcohol of the formula ROH wherein R represents a hydrocarbyl or substituted hydrocarbyl group having 1 to 20 carbon atoms.

In one preferred embodiment, the metal is divalent. Therefore, the invention also includes a process for the preparation of a metal carboxylate of the formula:

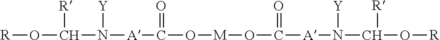

as defined above, characterised in that a metal carboxylate of the formula:

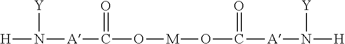

wherein each A′ represents an alkylene group having 1 to 6 carbon atoms; M represents a divalent metal ion; and Y represents a hydrocarbyl or substituted hydrocarbyl group having 1 to 20 carbon atoms; is reacted with an aldehyde of the formula R′CHO wherein R′ represents hydrogen or a hydrocarbyl or substituted hydrocarbyl group having 1 to 20 carbon atoms and an alcohol of the formula ROH wherein R represents a hydrocarbyl or substituted hydrocarbyl group having 1 to 20 carbon atoms.

The divalent metal ion M is preferably zinc. One example of a preferred zinc carboxylate according to the invention has the formula:

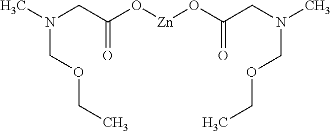

This zinc carboxylate can be prepared by the reaction of a zinc amino acid carboxylate of the formula (CH₃—NH—CH₂—COO)₂Zn with formaldehyde and ethanol.

The polymeric material containing carbon-to-carbon unsaturation can for example be a diene rubber. The diene elastomer can for example be natural rubber. The diene elastomer can alternatively be a synthetic polymer which is a homopolymer or copolymer of a diene monomer (a monomer bearing two double carbon-carbon bonds, whether conjugated or not). Preferably the elastomer is an “essentially unsaturated” diene elastomer, that is a diene elastomer resulting at least in part from conjugated diene monomers, having a content of members or units of diene origin (conjugated dienes) which is greater than 15 mol %. More preferably it is a “highly unsaturated” diene elastomer having a content of units of diene origin (conjugated dienes) which is greater than 50 mol %.

The diene elastomer can for example be:
(a) any homopolymer obtained by polymerization of a conjugated diene monomer having 4 to 12 carbon atoms;
(b) any copolymer obtained by copolymerization of one or more dienes conjugated together or with one or more vinyl aromatic compounds having 8 to 20 carbon atoms;
(c) a ternary copolymer obtained by copolymerization of ethylene, of an α-olefin having 3 to 6 carbon atoms with a non-conjugated diene monomer having 6 to 12 carbon atoms, such as, for example, the elastomers obtained from ethylene, from propylene with a non-conjugated diene monomer of the aforementioned type, such as in particular 1,4-hexadiene, ethylidene norbornene or dicyclopentadiene;
(d) a copolymer of isobutene and isoprene (butyl rubber), and also the halogenated, in particular chlorinated or brominated, versions of this type of copolymer.

Suitable conjugated dienes include 1,3-butadiene, 2-methyl-1,3-butadiene, 2,3-di(Ci-C5 alkyl)-1,3-butadienes such as, for instance, 2,3-dimethyl-1,3-butadiene, 2,3-diethyl-1,3-butadiene, 2-methyl-3-ethyl-1,3-butadiene, 2-methyl-3-isopropyl-1,3-butadiene, an aryl-1,3-butadiene, 1,3-pentadiene and 2,4-hexadiene.

Suitable vinyl aromatic compounds are, for example, styrene, ortho-, meta- and para-methylstyrene, the commercial mixture “vinyltoluene”, para-tert.-butylstyrene, methoxystyrenes, chlorostyrenes, vinylmesitylene, divinylbenzene and vinylnaphthalene. The copolymers may contain between 99% and 20% by weight of diene units and between 1% and 80% by weight of vinyl aromatic units. The elastomers may have any microstructure, which is a function of the polymerization conditions used, in particular of the presence or absence of a modifying and/or randomizing agent and the quantities of modifying and/or randomizing agent used. The elastomers may for example be block, statistical, sequential or microsequential elastomers, and may be prepared in dispersion or in solution; they may be coupled and/or starred or alternatively functionalized with a coupling and/or starring or functionalizing agent. Examples of preferred block copolymers are styrene-butadiene-styrene (SBS) block copolymers and styrene-ethylene/butadiene-styrene (SEBS) block copolymers.

The elastomer can be an alkoxysilane-terminated diene polymer or a copolymer of the diene and an alkoxy-containing molecule prepared via a tin coupled solution polymerization.

The amine compound of formula (I) can be used as the only crosslinking agent for the diene elastomer or can be used in conjunction with a known curing agent for the elastomer composition, for example be a conventional sulfur vulcanizing agent.

The amine compound of formula (I), particularly a substituted piperazine, can alternatively be incorporated in a diene elastomer composition, particularly a natural rubber composition used in tyres, as an anti-reversion agent. An anti-reversion agent is an agent used in natural rubber to “heal” and cure the rubber while it is degrading with high temperature (160° C.). Heat durability of a tire tread is often a factor for vehicular tires intended to be driven at relatively high speeds. Heat is inherently generated within a tire tread rubber compound as the tire is driven at relatively high speeds resulting in a temperature rise within the tire tread itself.

It is desired to reduce the rate of temperature rise within a sulfur cured tire tread rubber composition with an attendant increase in its heat durability. Incorporation of an amine compound of formula (I) particularly a substituted piperazine, in the tread rubber composition retards the rate of temperature rise within the tread rubber composition.

When the amine compound of formula (I) is incorporated in a sulfur cured tire tread rubber composition as an anti-reversion agent, the amine compound of formula (I) can for example be added with the vulcanization system. The rubber compositions are preferably produced using the conventional two successive preparation phases of mechanical or thermo-mechanical mixing or kneading (“non-productive” phase) at high temperature, followed by a second phase of mechanical mixing (“productive” phase) at lower temperature, typically less than 110° C., for example between 40° C. and 100° C., during which the vulcanization system is incorporated. If the amine compound of formula (I) is incorporated in the rubber composition at this lower temperature, it does not act significantly as a crosslinking agent during production of the cured rubber, but remains in the rubber composition to act as an anti-reversion agent.

The polymeric material containing carbon-to-carbon unsaturation can alternatively be an organopolysiloxane containing alkenyl groups. Examples of alkenyl groups of the organopolysiloxane include vinyl, allyl, butenyl, pentenyl, hexenyl, and heptenyl groups, of which vinyl groups are preferred. Silicon-bonded organic groups other than alkenyl groups contained in the organopolysiloxane may be exemplified by methyl, ethyl, propyl, butyl, pentyl, hexyl, or similar alkyl groups; phenyl, tolyl, xylyl, or similar aryl groups; or 3-chloropropyl, 3,3,3-trifluoropropyl, or similar halogen-substituted groups. Preferably, the groups other than alkenyl groups are methyl groups and optionally phenyl groups.

For many uses it is preferred that the major part of the organopolysiloxane has a predominantly linear molecular structure, such as a polydiorganosiloxane. The organopolysiloxane can for example comprise an α,ω-vinyldimethylsiloxy polydimethylsiloxane, an α,ω-vinyldimethylsiloxy copolymer of methylvinylsiloxane and dimethylsiloxane units, and/or an α,ω-trimethylsiloxy copolymer of methylvinylsiloxane and dimethylsiloxane units.

The organopolysiloxane can additionally or alternatively comprise a branched organopolysiloxane containing alkenyl units. Such a branched organopolysiloxane can for example comprise ViSiO_{3/2} (where Vi represents vinyl), CH₃SiO_{3/2} and/or SiO_{4/2} branching units with (CH₃₎₂Vi SiO_{1/2} and/or (CH₃₎₃SiO_{1/2} and optionally CH₃Vi SiO_{2/2} and/or (CH₃₎₂SiO_{2/2} units, provided that at least one vinyl group is present. A branched organopolysiloxane can for example consist of (i) one or more Q units of the formula(SiO_{4/2}) and (ii) from 15 to 995 D units of the formula R^{b}₂SiO_{2/2}, which units (i) and (ii) may be inter-linked in any appropriate combination, and M units of the formula R^{a}R^{b}₂SiO_{1/2}, wherein each R^{a} substituent is selected from the group consisting of an alkyl group having from 1 to 6 carbon atoms, an alkenyl group having from 1 to 6 carbon atoms and an alkynyl group having from 1 to 6 carbon atoms, at least three Ra substituents in the branched siloxane being alkenyl or alkynyl units, and each R^{b} substituent is selected from the group consisting of an alkyl group having from 1 to 6 carbon atoms, an alkenyl group having 2 to 6 carbon atoms, an aryl group, an alkoxy group, an acrylate group and a methacrylate group, as described in U.S. Pat. No. 6,806,339.

The polyorganosiloxane can for example have a viscosity of at least 100 mPa·s at 25° C., preferably at least 300 mPa·s, and may have a viscosity of up to 90000 mPa·s, preferably up to 70000 mPa·s.

Organopolysiloxanes containing alkenyl groups are used for example in release coating compositions for paper and other substrates, and in liquid silicone rubber compositions used for coating air bags and for other applications. The amine compound of formula (I), particularly a substituted piperazine, can be used as all or part of the crosslinking agent in such compositions.

### EXAMPLES

Crosslinker 1 has been prepared following the article in Russian Journal of Applied Chemistry; Volume 82, Issue 5, Pages 928-930; Journal 2009; by V. M. Farzaliev, M. T. Abbasova, A. A. Ashurova, G. B. Babaeva, N. 15 P. Ladokhina and Ya. M. Kerimova which describes the preparation of bis(alkoxymethyl)piperazines by condensation of piperazine with formaldehyde and aliphatic alcohols.

### Example 1 to 4

Rubber goods were prepared according to the procedure described below for example 1 to 4.

The amounts expressed in parts per hundred parts of rubber (phr) are described in table 1.
NR SMR 10, CV60—Natural rubber Technical Standard Rubber, purity grade 10, Constant viscosity (CV) 60 m.u. (Mooney unit)
Silica—Zeosil® 1165MP from Rhodia
Silane 1—Bis-(triethoxysilylpropyl)-tetrasulfane—Z-6940 by Dow Corning
ACST—Stearic Acid
ZnO—Zinc Oxide
6PPD—N-1,3-dimethylbutyl-N-phenyl-para-phenylenediamine from Rhein Chemie
DPG 80%—diphenylguanidine supported on EPDM at 80% active material from Rhein Chemie (Vulkanox® 4020/LG)
Crosslinker 1—N,N′-diethoxy-methyl-piperazine

| | Example | | | |
|---|---|---|---|---|
| | 1 | 2 | 3 | 4 |
| NR SMR 10 CV60 | 100.00 | 100.00 | 100.00 | 100.00 |
| Silica-Z-1165MP | 60.00 | 60.00 | 60.00 | 60.00 |
| Silane 1 | 6.00 | 6.00 | 6.00 | 6.00 |
| ZnO | 3.00 | 0.00 | 0.00 | 0.00 |
| AcSt | 2.50 | 0.00 | 0.00 | 0.00 |
| 6PPD | 2.00 | 0.00 | 0.00 | 0.00 |
| AcSt | 0.00 | 0.00 | 0.00 | 2.00 |
| Crosslinker 1 | 4.00 | 4.00 | 4.00 | 4.00 |
| Rhenocure DPG 80% | 0.50 | 0.00 | 2.00 | 0.00 |

During a first non-productive phase, the reaction of the natural rubber, filler and when present silane was carried out using thermomechanical kneading in a Banbury mixer. The procedure was as shown in Table 2, which indicates the time of addition of various ingredients. The-temperature at the end of mixing was measured inside the rubber after dumping it from the mixer.

TABLE 1

| | Time (seconds) | | | | |
|---|---|---|---|---|---|
| | 0 | 60 | 90 | 150 | 360 |
| Ingredient | Natural | ⅔ Filler | ⅓ filler | Ram opening | End |
| | rubber | (Silane) | | | mixing |
| Mixer internal | 80 | 90 | 100 | 160 | 155-165 |
| probe | | | | | |
| indicative | | | | | |
| temperature | | | | | |
| (° C.) | | | | | |

During a second non-productive phase stearic acid, zinc oxide and 6PPD were added to the obtained compound from the first non-productive phase. The mixing was carried out using thermomechanical kneading in a Banbury mixer. The procedure was as shown in Table 3, which indicates the time of addition of various ingredients and the estimated temperature of the mixture at that time.

TABLE 2

| | Time (seconds) | | |
|---|---|---|---|
| | 0 | 30 | 300 |
| Ingredient | Natural rubber | ZnO | End |
| | | AcSt | mixing |
| | | 6PPD | |
| Mixer internal probe | 80 | 90 | 155-165 |
| indicative | | | |
| temperature (° C.) | | | |

The modified natural rubber composition thus produced was milled on a two-roll mill at a temperature of about 70° C. during which milling the curing agents were added (productive phase). The mixing procedure for the productive phase is shown in Table 4.

TABLE 3

| | Number | Roll | |
|---|---|---|---|
| 2 roll mill | of | distance | |
| process step | passes | (mm) | Time/action |
| Heating up rubber | 5 | 4.0 | NA |
| | 1 | 3.5 | NA |
| | 1 | 3.0 | NA |
| | 1 | 2.5 | NA |
| Mixing rubber | NA | 2-2.4 | Form a mantle around one roll |
| and additives | | | add curing additives |
| | | | within 2.0 minutes |
| | | | cut and turn sheet regularly |
| | | | Stop after 6.0 minutes |
| Sheet formation | 3 | 2.5 | roll up |
| | 2 | 5.1 | Roll on first pass 3-ply for second |
| | 1 | 2.3-2.5 | For final sheet for cutting, |
| | | | moulding and curing |

The modified rubber sheet produced was tested as follows. The results of the tests are shown in Table X below.

The rheometry measurements were performed at 160° C. using an oscillating chamber rheometer (i.e., Advanced Plastic Analyzer) in accordance with Standard ISO 3417:1991 (F). The change in rheometric torque over time describes the course of stiffening of the composition as a result of the vulcanization reaction. The measurements are processed in accordance with Standard ISO 3417:1991(F). Minimum and maximum torque values, measured in deciNewtonmeter (dNm) are respectively denoted ML and MH time at α% cure (for example 5%) is the time necessary to achieve conversion of α% (for example 5%) of the difference between the minimum and maximum torque values. The difference, denoted MH-ML, between minimum and maximum torque values is also measured. In the same conditions the scorching time for the rubber compositions at 160° C. is determined as being the time in minutes necessary to obtain an increase in the torque of 2 units, above the minimum value of the torque (‘Time@2dNm scorch S’).

TABLE 4

Example

| | 1 | 2 | 3 | 4 |
|---|---|---|---|---|
| ML | 1.65 | 1.74 | 1.56 | 1.38 |
| MH | 2.62 | 3.56 | 3.36 | 2.91 |
| MH − ML | 0.96 | 1.82 | 1.80 | 1.54 |

Crosslinker 1 showed small level of crosslinking based on increased MH-ML. Additives classically used in rubber compound formulation were not able to accelerate curing speed and to increase crosslinking density of compound.

### Prophetic Example

Crosslinker 1 had reacted with Natural rubber through liberation of ethanol form the ethoxy-methyl amine part and by removal of a proton in alpha to the nitrogen within the piperazine cycle.

To improve reactivity of crosslinker 1 catalytic system will be used like Lewis Acid or strong base typically used in SBR or BR synthesis as for example cited in patent WO2005/085343,

A second possibility is to increase the distance between the 2 reactive sites or by having proton in alpha to the nitrogen outside of a cycle as for example using the following structure as secondary amine raw material:

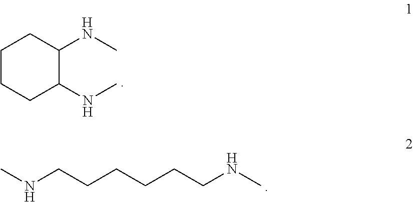

In case of raw material 1 the proton abstraction will occur on the CH3 and both reactive site will not affect the other.

Similarly in case of molecule site the proton abstraction will occur on the outside CH2-CH3 group and both group will not affect the other.

Similarly to crosslinker 1 alkoxy-methyl amine version will be prepared using an alcohol and para-formaldehyde. The reaction will form the following species:

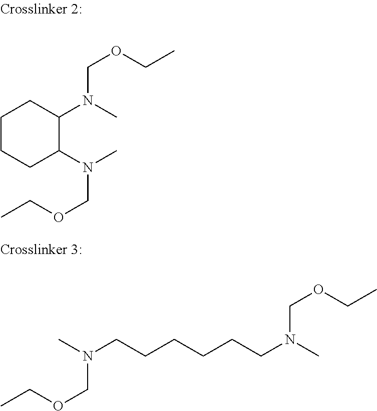 [SMILES: CCOCN(C)C1CCCCC1N(C)COCC ; CCOCN(C)CCCCCCN(C)COCC]

Reinforced rubber using silica/silane as reinforcing system will be prepared as described previously in example 1 to 4 and will be tested according to same procedure as for crosslinker

Similarly to crosslinker 1, crosslinker 4 will be prepared using butane-diol molecule to create a polymeric structure. This structure will help to increase distance between reactive site and will increase crosslinking capability. Reinforced rubber using silica/silane as reinforcing system will be prepared as described previously in example 1 to 4 and will be tested according to same procedure as for crosslinker

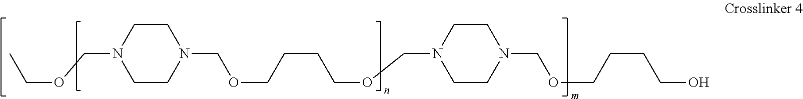

1. A process for modifying a polymeric material containing reactive carbon-to-carbon unsaturation, characterised in that the polymeric material is treated with a compound (I) containing in its molecule at least two moieties of the formula 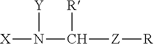
wherein X represents a hydrocarbyl or substituted hydrocarbyl group having 1 to 20 carbon atoms; Y represents hydrogen or a hydrocarbyl or substituted hydrocarbyl group having 1 to 20 carbon atoms; R′ represents hydrogen or a hydrocarbyl or substituted hydrocarbyl group having 1 to 20 carbon atoms; Z represents oxygen or sulphur; and R represents a hydrocarbyl or substituted hydrocarbyl group having 1 to 20 carbon atoms, at least one of the groups X and R being a multivalent substituted hydrocarbyl group linking two or more 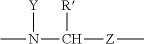
moieties.

2. A process according to claim 1, characterised in that the groups X and Y are both substituted hydrocarbyl groups linking the same two 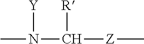
moieties to form a piperazine ring.

3. A process according to claim 2, characterised in that the compound (I) is a substituted piperazine of the formula [R—Z—(CHR′-Pip-CHR′—Z—R″—Z)ₙ—CH₂-Pip-CH₂—Z]ₘ—R*, where each R represents a hydrocarbyl or substituted hydrocarbyl group having 1 to 20 carbon atoms; each R′ represents hydrogen or a hydrocarbyl or substituted hydrocarbyl group having 1 to 8 carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=1 to 6; and R* is the residue of an alcohol, thiol, polyol or polythiol having at least m hydroxyl or thiol groups.

4. A process according to claim 3, characterised in that n=0, m=1, each atom Z in the substituted piperazine represents an oxygen atom and each group R represents a hydrocarbyl group having 1 to 8 carbon atoms.

5. A process according to claim 4, characterised in that R* represents the residue of a polyol selected from ethylene glycol, propylene glycol, 1,4-butanediol, trimethylolpropane and pentaerythritol.

6. A process according to claim 1, characterised in that X represents a multivalent substituted hydrocarbyl group linking two or more 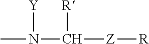
groups; Y represents a hydrocarbyl or substituted hydrocarbyl group having 1 to 20 carbon atoms; Z represents oxygen; and none of Y, R and R′ is a multivalent substituted hydrocarbyl group linking two or more 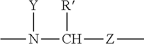
Moieties to form compound (I)

7. A process according to claim 6, characterised in that the compound (I) has the formula 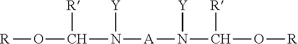
wherein each R, R′ and Y is defined as in claim 6 and A represents a divalent organic group having 2 to 20 carbon atoms.

8. A process according to claim 6, characterised in that the compound (I) has the formula 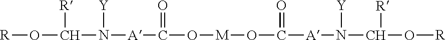
wherein each R, R′ and Y is defined as in claim 6; each A′ represents an alkylene group having 1 to 6 carbon atoms; and M represents a divalent metal ion.

9. A process according to claim 1, characterised in that R represents a multivalent substituted hydrocarbyl group linking two or more 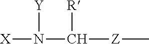
groups; Y represents a hydrocarbyl or substituted hydrocarbyl group having 1 to 20 carbon atoms; and none of X, Y, and R′ is a multivalent substituted hydrocarbyl group linking two or more 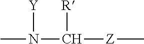
moieties.

10. A process according to claim 1 wherein the polymeric material and the compound (I) are heated together at a temperature of 120 to 200° C., whereby the polymeric material is crosslinked by the compound (I) or wherein the polymeric material and the compound (I) are mixed at a temperature of 0 to 120° C. and subsequently heated at a temperature of 120 to 200° C. to crosslink the polymeric material.

11. (canceled)

12. A process according to claim 1, characterised in that the polymeric material is a diene rubber, the polymeric material is an organopolysiloxane containing alkenyl groups, or the polymeric material is a diene rubber and the polymeric material is an organopolysiloxane containing alkenyl groups.

13. (canceled)

14. A substituted piperazine of the formula [R—Z—(CHR′-Pip-CHR′—Z—R″—Z)ₙ—CH₂-Pip-CH₂—Z]ₘ—R*, where each R represents a hydrocarbyl or substituted hydrocarbyl group having 2 to 20 carbon atoms; each R′ represents hydrogen or a hydrocarbyl or substituted hydrocarbyl group having 1 to 8 carbon atoms; Pip represents an optionally substituted piperazine ring bonded through its nitrogen atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=2 to 6; and R* is the residue of a polyol or polythiol having at least m hydroxyl or thiol groups.

15. A substituted piperazine according to claim 14, characterised in that each R′ represents a hydrogen atom, the 2-, 3-, 5- and 6-positions on the piperazine ring are unsubstituted, or each R′ represents a hydrogen atom and the 2-, 3-, 5- and 6-positions on the piperazine ring are unsubstituted.

16. (canceled)

17. A substituted piperazine according to claim 14, characterised in that R* is the residue of a polyol selected from ethylene glycol, propylene glycol, 1,4-butanediol, trimethylolpropane and pentaerythritol.

18. A process for the preparation of a substituted piperazine of the formula 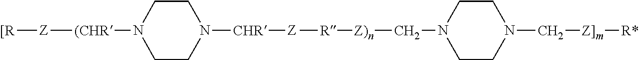
where each R represents a hydrocarbyl or substituted hydrocarbyl group having 1 to 20 carbon atoms; each R′ represents hydrogen or a hydrocarbyl or substituted hydrocarbyl group having 1 to 8 carbon atoms; each Z represents an oxygen or sulphur atom; R″ represents an alkylene, hydroxyalkylene, thioalkylene or polyoxyalkylene linkage having 2 to 20 carbon atoms or an alkylene, hydroxyalkylene, thioalkylene or polyoxyalkylene linkage having 2 to 20 carbon atoms substituted by 1 to 4 R—Z—CHR′-Pip-CHR′—Z— groups, where R, R′, Z and Pip are defined as above; n=0 to 20; m=2 to 6; and R* is the residue of an alcohol or polyol having at least m hydroxyl groups, characterised in that piperazine is reacted with an aldehyde of the formula R′CHO and a polyol or polythiol of the formula R*(ZH)_{z}, where z=2 to 6 and z is greater than or equal to m.

19. A process according to claim 18 characterised in that the piperazine and the aldehyde are reacted with a mixture of a polyol of the formula R*(OH)_{z} and an alcohol of the formula ROH, where R represents a hydrocarbyl group having 1 to 20 carbon atoms.

20. A Metal carboxylate of the formula M(-O—C(═O)-A′-N(Y)—CH(R′)—O—R)ₘ wherein each A′ represents an alkylene group having 1 to 6 carbon atoms; M represents a metal ion of charge m; and Y represents a hydrocarbyl or substituted hydrocarbyl group having 1 to 20 carbon atoms; is reacted with an aldehyde of the formula R′CHO wherein R′ represents hydrogen or a hydrocarbyl or substituted hydrocarbyl group having 1 to 20 carbon atoms and an alcohol of the formula ROH wherein R represents a hydrocarbyl or substituted hydrocarbyl group having 1 to 20 carbon atoms, preferably a metal carboxylate of the formula 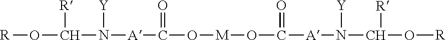
wherein each A′ represents an alkylene group having 1 to 6 carbon atoms; M represents a divalent metal ion; R represents a hydrocarbyl or substituted hydrocarbyl group having 1 to 20 carbon atoms; R′ represents hydrogen or a hydrocarbyl or substituted hydrocarbyl group having 1 to 20 carbon atoms; Y represents a hydrocarbyl or substituted hydrocarbyl group having 1 to 20 carbon atoms; and none of Y, R and R′ is a multivalent substituted hydrocarbyl group linking two or more 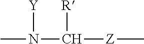
moieties.

21. A metal carboxylate according to claim 20 wherein the divalent metal is zinc.

22. A zinc carboxylate according to claim 21 having the formula

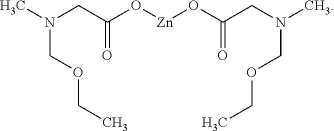

23. A process for the preparation of a metal carboxylate of the formula 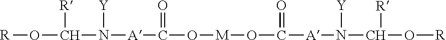
as defined in claim 20, characterised in that a metal carboxylate of the formula 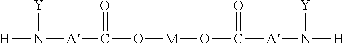
wherein each A′ represents an alkylene group having 1 to 6 carbon atoms; M represents a divalent metal ion; and Y represents a hydrocarbyl or substituted hydrocarbyl group having 1 to 20 carbon atoms; is reacted with an aldehyde of the formula R′CHO wherein R′ represents hydrogen or a hydrocarbyl or substituted hydrocarbyl group having 1 to 20 carbon atoms and an alcohol of the formula ROH wherein R represents a hydrocarbyl or substituted hydrocarbyl group having 1 to 20 carbon atoms.

24-25. (canceled)
